(19) United States
(12) Patent Application Publication
(10) Pub. No.: US 2026/0216474 A1
(43) Pub. Date: Jul. 30, 2026

(54) DIFFUSION CATHETER SYSTEMS FOR DELIVERY OF FLUID-BASED THERAPIES

(71) Applicant: Boston Scientific Scimed, Inc., Maple Grove, MN (US)

(72) Inventors: Daniel K. Tomaschko, Savage, MN (US); Ajay Gupta, Minneapolis, MN (US); Timothy A. Ostroot, Cokato, MN (US); Mark Steven Smith, Coon Rapids, MN (US); Robert M. Wold, Maple Lake, MN (US)

(73) Assignee: MAYO FOUNDATION FOR MEDICAL EDUCATION AND RESEARCH, Rochester, MN (US)

(21) Appl. No.: 19/461,179

(22) Filed: Jan. 27, 2026

Related U.S. Application Data
(60) Provisional application No. 63/751,623, filed on Jan. 30, 2025.

(51) Int. Cl. A61M 25/00

(52) U.S. Cl. CPC A61M 25/0021; A61M 25/005; A61M 25/007; A61M 25/0075; A61M 2025/0042; A61M 2025/0073; A61M 2025/0076; A61M 2025/0079; A61M 25/0097; A61M 2202/0007; A61M 2202/04; A61M 2205/3331; A61M 2206/20; A61M 2210/12

(57) ABSTRACT

Embodiments herein relate to devices for delivering fluid-based therapies into a target vessel of a patient. In an embodiment, an apparatus is included having a microcatheter. The microcatheter can include a catheter shaft having a lumen therein. The microcatheter can have an open end that is at a distal end of the catheter shaft. The microcatheter can have a plurality of side apertures disposed within a side wall of the catheter shaft. The plurality of side apertures allow for a fluid to pass from the lumen to the outside of the catheter shaft. The apparatus further includes a seal element configured to pass through the lumen of the microcatheter. The seal element can include a shaft and an occlusion member configured to at least partially occlude the open end of the microcatheter directing fluid flow through the plurality of side apertures. Other embodiments are also included herein.

This application claims the benefit of U.S. Provisional Application No. 63/751,623 filed January 30, 2025, the content of which is herein incorporated by reference in its entirety.

### FIELD

Embodiments herein relate to diffusion systems including catheters, insert wires, and the like used to provide therapies to a target vessel of a patient and related methods.

### BACKGROUND

Catheter-based technologies can be used for the targeted delivery of fluid-based therapies, including chemotherapeutic agents, radioactive microspheres in a suspension, and other therapeutic compositions, to specific sites within the human body. Conventionally, these delivery systems are designed to access constrained anatomical structures like blood vessels. Such catheter systems typically include a flexible elongated tube with a distal tip through which the fluid-based therapy is delivered. Traditional designs often rely on a single distal opening for fluid emission.

### SUMMARY

Embodiments herein relate to diffusion systems including catheters, insert wires, and the like used to provide therapies to a target vessel of a patient and related methods. In a first aspect, an apparatus for delivering fluid-based therapies can be included having a microcatheter. The microcatheter can include a catheter shaft and a lumen, wherein the lumen can be within the catheter shaft. The catheter shaft can include an open end at a distal end of the catheter shaft and a plurality of side apertures. The plurality of side apertures can be disposed within a side wall of the catheter shaft and can allow for a fluid to pass from the lumen to the outside of the catheter shaft. The apparatus can also include a seal element, wherein the seal element can be configured to pass through the lumen of the microcatheter. The seal element can include a shaft and an occlusion member. The occlusion member can be attached to the shaft and can be configured to at least partially occlude the open end of the microcatheter directing fluid flow through the plurality of side apertures.

In a second aspect, in addition to one or more of the preceding or following aspects, or in the alternative to some aspects, the microcatheter can further include a distal sealing feature.

In a third aspect, in addition to one or more of the preceding or following aspects, or in the alternative to some aspects, the distal sealing feature can include a tapered portion, wherein the tapered portion can be disposed adjacent the open end and narrows the lumen.

In a fourth aspect, in addition to one or more of the preceding or following aspects, or in the alternative to some aspects, the occlusion member can be configured to engage with the tapered portion.

In a fifth aspect, in addition to one or more of the preceding or following aspects, or in the alternative to some aspects, the distal sealing feature can include a stepped down portion.

In a sixth aspect, in addition to one or more of the preceding or following aspects, or in the alternative to some aspects, the distal sealing feature can include an internal bump.

In a seventh aspect, in addition to one or more of the preceding or following aspects, or in the alternative to some aspects, the occlusion member can include a stopper.

In an eighth aspect, in addition to one or more of the preceding or following aspects, or in the alternative to some aspects, the occlusion member can include a textured surface, wherein the textured surface can be configured to enhance engagement with an interior surface of the open end of the microcatheter.

In a ninth aspect, in addition to one or more of the preceding or following aspects, or in the alternative to some aspects, the plurality of side apertures can allow for diffusion and/or homogenization of the therapy within a target vessel after passing out of the microcatheter.

In a tenth aspect, in addition to one or more of the preceding or following aspects, or in the alternative to some aspects, the plurality of side apertures can be in the form of slots or round holes.

In an eleventh aspect, in addition to one or more of the preceding or following aspects, or in the alternative to some aspects, the plurality of side apertures can be configured to change shape and/or size at a threshold pressure.

In a twelfth aspect, in addition to one or more of the preceding or following aspects, or in the alternative to some aspects, the plurality of side apertures can be arranged around the circumference of the microcatheter.

In a thirteenth aspect, in addition to one or more of the preceding or following aspects, or in the alternative to some aspects, the shaft of the seal element can be a wire shaft.

In a fourteenth aspect, in addition to one or more of the preceding or following aspects, or in the alternative to some aspects, a portion of the shaft extends beyond the occlusion member.

In a fifteenth aspect, in addition to one or more of the preceding or following aspects, or in the alternative to some aspects, the portion of the shaft extending beyond the stopper includes a steering element configured to aid in repositioning of the microcatheter.

In a sixteenth aspect, in addition to one or more of the preceding or following aspects, or in the alternative to some aspects, the microcatheter can be configured to be repositionable within a vessel of a subject to allow for movement to multiple therapy delivery sites without removal of the seal element from the microcatheter.

In a seventeenth aspect, in addition to one or more of the preceding or following aspects, or in the alternative to some aspects, the microcatheter can further include a proximal end, wherein the proximal end can be configured to connect to a fluid therapy source.

In an eighteenth aspect, in addition to one or more of the preceding or following aspects, or in the alternative to some aspects, can further include a locking mechanism, wherein the locking mechanism can be configured to secure the seal element in place within the microcatheter.

In a nineteenth aspect, in addition to one or more of the preceding or following aspects, or in the alternative to some aspects, the catheter shaft can include a shaft wall with a non-braided structure disposed within the shaft wall.

In a twentieth aspect, in addition to one or more of the preceding or following aspects, or in the alternative to some aspects, the catheter shaft can include a shaft wall with a braided structure disposed within the shaft wall. In a twenty-first aspect, in addition to one or more of the preceding or following aspects, or in the alternative to some aspects, the plurality of side apertures can be disposed within open gaps of the braided structure.

In a twenty-second aspect, in addition to one or more of the preceding or following aspects, or in the alternative to some aspects, the seal element can further include one or more radiopaque markers.

In a twenty-third aspect, in addition to one or more of the preceding or following aspects, or in the alternative to some aspects, the seal element can further include one or more alignment marks on a proximal end thereof.

In a twenty-fourth aspect, in addition to one or more of the preceding or following aspects, or in the alternative to some aspects, the microcatheter can further include one or more alignment marks on a proximal end thereof.

In a twenty-fifth aspect, in addition to one or more of the preceding or following aspects, or in the alternative to some aspects, the apparatus can further include a releasable locking mechanism, wherein the releasable locking mechanism can be configured to releasably secure the microcatheter to a seal wire in a selected position.

In a twenty-sixth aspect, in addition to one or more of the preceding or following aspects, or in the alternative to some aspects, the apparatus for delivering fluid-based therapies can further include a proximal fitting and a hypotube, wherein the hypotube can be connected to the proximal fitting and the seal element. The hypotube can be configured to receive the fluid and guide the same at least partway through the microcatheter.

In a twenty-seventh aspect, in addition to one or more of the preceding or following aspects, or in the alternative to some aspects, the apparatus is configured so that the fluid passes through the hypotube and to the lumen of the microcatheter and then through the plurality of side apertures.

In a twenty-eighth aspect, in addition to one or more of the preceding or following aspects, or in the alternative to some aspects, the proximal fitting can be a Luer fitting.

In a twenty-ninth aspect, a method of delivering a fluid-based therapy into a vessel of a patient can be included. The method can include inserting a microcatheter into the vessel of the patient, wherein the microcatheter includes a plurality of side apertures near its distal end. The method can further include inserting a seal wire into the microcatheter, wherein the seal wire includes a seal element. The method can further include advancing the seal wire to engage the seal element with a portion of a distal end of the microcatheter, locking the seal wire to the microcatheter, and delivering therapy fluid through the microcatheter and out the side apertures into the vessel.

In a thirtieth aspect, in addition to one or more of the preceding or following aspects, or in the alternative to some aspects, the method can further include repositioning the microcatheter and seal wire within the vessel for delivery of therapy to multiple sites without removing the seal wire from the microcatheter.

In a thirty-first aspect, in addition to one or more of the preceding or following aspects, or in the alternative to some aspects, the fluid-based therapy includes a cancer therapy.

In a thirty-second aspect, in addition to one or more of the preceding or following aspects, or in the alternative to some aspects, the fluid-based therapy includes radioactive microspheres in a carrier fluid.

In a thirty-third aspect, an insert wire for delivering fluid-based therapies to a vessel of a patient can be included having a flexible hollow shaft, and a diffusion tip. The diffusion tip can be at a distal end of the flexible hollow shaft. The diffusion tip can include a plurality of side apertures and an at least partially closed distal end. A connection hub can be disposed at a proximal end of the insert wire wherein the connection hub can be configured to allow connection of the insert wire to a fluid source.

In a thirty-fourth aspect, in addition to one or more of the preceding or following aspects, or in the alternative to some aspects, the diffusion tip can be configured to cause diffusion and/or homogenization of the therapy within a target vessel after passing out of the insert wire.

In a thirty-fifth aspect, in addition to one or more of the preceding or following aspects, or in the alternative to some aspects, the flexible hollow shaft can be a hypotube.

In a thirty-sixth aspect, in addition to one or more of the preceding or following aspects, or in the alternative to some aspects, the flexible hollow shaft can be a metal hypotube.

In a thirty-seventh aspect, in addition to one or more of the preceding or following aspects, or in the alternative to some aspects, the flexible hollow shaft can be a metal hypotube defining surface features to facilitate flexibility.

In a thirty-eighth aspect, in addition to one or more of the preceding or following aspects, or in the alternative to some aspects, the surface features can include slots, wherein the slots can be configured to aid with pushability and/or torque.

In a thirty-ninth aspect, in addition to one or more of the preceding or following aspects, or in the alternative to some aspects, the flexible hollow shaft includes a combination of a polymeric inner material and a metallic outer hypotube.

In a fortieth aspect, in addition to one or more of the preceding or following aspects, or in the alternative to some aspects, the flexible hollow shaft includes a metallic coil or metallic braided material in combination with a polymeric tube.

In a forty-first aspect, in addition to one or more of the preceding or following aspects, or in the alternative to some aspects, the insert wire can be configured to be inserted into a microcatheter in place of a standard guidewire.

In a forty-second aspect, in addition to one or more of the preceding or following aspects, or in the alternative to some aspects, the plurality of side apertures can be in the form of slots or round holes.

In a forty-third aspect, in addition to one or more of the preceding or following aspects, or in the alternative to some aspects, the plurality of side apertures can be configured to change shape and/or size at a threshold pressure.

In a forty-fourth aspect, in addition to one or more of the preceding or following aspects, or in the alternative to some aspects, the plurality of side apertures can be spaced around the circumference of the diffusion tip.

In a forty-fifth aspect, in addition to one or more of the preceding or following aspects, or in the alternative to some aspects, can further include a steering element, wherein the steering element extends beyond the at least partially closed distal end.

In a forty-sixth aspect, in addition to one or more of the preceding or following aspects, or in the alternative to some aspects, the insert wire allows repositioning of a microcatheter without removal of the insert wire.

In a forty-seventh aspect, in addition to one or more of the preceding or following aspects, or in the alternative to some aspects, the at least partially closed distal end can be configured to minimize dead space at the end of the flexible hollow shaft.

In a forty-eighth aspect, in addition to one or more of the preceding or following aspects, or in the alternative to some aspects, can further include one or more radiopaque markers.

In a forty-ninth aspect, in addition to one or more of the preceding or following aspects, or in the alternative to some aspects, the distal end can be fully closed.

In a fiftieth aspect, in addition to one or more of the preceding or following aspects, or in the alternative to some aspects, the flexible hollow shaft can include a reinforcement layer, wherein the reinforcement layer can be configured to prevent kinking.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures(FIGS.), in which:

FIG. 1 is a schematic view of vessel anatomy relevant for injectate delivery in accordance with various embodiments herein.

FIG. 2 is a schematic view of a microcatheter in accordance with various embodiments herein.

FIG. 3 is a schematic view of a seal element in accordance with various embodiments herein.

FIG. 4 is a cross-sectional view of a distal portion of a therapy delivery apparatus in accordance with various embodiments herein.

FIG. 5 is a schematic view of a microcatheter within vessel anatomy in accordance with various embodiments herein.

FIG. 6 is a schematic view of an apparatus herein within vessel anatomy in accordance with various embodiments herein.

FIG. 7 is a schematic view of an apparatus herein within vessel anatomy in accordance with various embodiments herein.

FIG. 8 is a schematic view of an apparatus herein within vessel anatomy in accordance with various embodiments herein.

FIG. 9 is a schematic view of an apparatus herein within vessel anatomy in accordance with various embodiments herein.

FIG. 10 is a schematic view of a microcatheter wall in accordance with various embodiments herein.

FIG. 11 is a schematic view of an insert wire in accordance with various embodiments herein.

FIG. 12 is a schematic cross-sectional view of a distal end of an insert wire in accordance with various embodiments herein.

FIG. 13 is a schematic cross-sectional view of a distal end of an insert wire in accordance with various embodiments herein.

FIG. 14 is a schematic cross-sectional view of a distal end of an insert wire in accordance with various embodiments herein.

FIG. 15 is a schematic partial cross-sectional view of a therapy delivery apparatus in accordance with various embodiments herein.

FIG. 16 is a schematic partial cross-sectional view of a therapy delivery apparatus in accordance with various embodiments herein.

FIG. 17 is a flow chart of operations of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Traditional designs for catheters used to deliver fluids often rely on a single distal opening for fluid emission. However, direct flow exiting a single distal opening can lead to a stream or “jetting” of therapy within the vessel space. This can lead to a non-homogenous distribution of the therapeutic composition within the vessel and potentially inadequate therapeutic exposure across the target site(s) particularly in complex vascular networks and for therapies requiring precise localized delivery. This can be particularly critical in treatments involving tissue-specific targets, where precise dosage and even distribution can significantly influence therapeutic outcomes.

The present invention provides an advanced diffusion catheter system designed to overcome the limitations of traditional catheter-based fluid delivery methods by enabling superior diffusion characteristics and enhanced positional flexibility including the ability to reposition the catheter to target multiple sites without creating potential contamination issues. In some embodiments, diffusion catheters herein can include a microcatheter with a plurality of side apertures along its catheter shaft. A seal element can be configured to pass through the lumen of the microcatheter. This seal element can include a shaft and an occlusion member, which is positioned to partially or entirely block an open distal end of the microcatheter. By blocking (at least partially) flow through the open distal end, fluid flow is redirected through the side apertures allowing for more uniform distribution and diffusion of therapeutic agent within the targeted vessel. These features are particularly beneficial for delivering treatments that require high precision and even distribution, such as chemotherapies and radioactive microspheres, especially in intricate vascular systems. Such controlled diffusion can enhance the efficacy of therapies that target specific tissue regions, promising improved patient outcomes with reduced systemic exposure.

Embodiments herein can further solve conventional issues related to catheter repositioning within a vessel without the need for complete withdrawal of certain elements thereof. For example, in many cases, repositioning a microcatheter may require maneuvering a guidewire into a desired position within the vasculature. However, normally, the guidewire is removed to allow therapy delivery, so if a clinician desires to reposition the microcatheter to treat another site, the guidewire must be reinserted (and exposed to the therapeutic materials that have passed through the microcatheter previously) and then removed. This can create issues, particularly where the therapeutic composition includes radioactive microparticles. However, the seal element, which may be locked in place via a releasable locking mechanism within the catheter, allows re-angling and repositioning of the microcatheter without separate reinsertion of a guidewire. This adaptive feature is particularly advantageous for delivering therapies to multiple sites in a single procedure, thus reducing potential contamination issues. Thus, a seal element with a steering component aids in maneuverability, paving the way for more dynamic procedural applications.

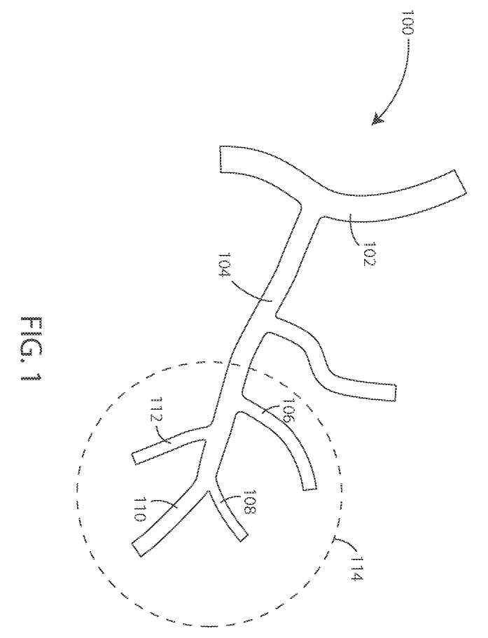

Referring now to FIG. 1, a schematic view is shown of vessel anatomy relevant for injectate delivery in accordance with various embodiments herein. In particular, FIG. 1 presents a schematic view of a complex vessel network as may be relevant for the use of diffusion catheters for fluid-based therapies herein. The structure is centered around a small diameter access vessel (102), which serves as the primary conduit into the illustrated branching network.

Extending from the access vessel (102) is a side branch vessel (104), directing flow toward multiple feeder vessels. These include the first feeder vessel (106), the second feeder vessel (108), the third feeder vessel (110), and the fourth feeder vessel (112). Each feeder vessel bifurcates or diverges from the side branch vessel (104). In this example, each feeder vessel may be feeding a tumor. An exemplary therapy target area (114) is marked by a dashed circle, and is the site intended for the focused delivery of therapeutic agents. The feeder vessel target area (114) highlights a region within the vessel network where enhanced diffusion and precise distribution of therapeutic substances can be beneficial for treatment. The layout shown in FIG. 1 is indicative of the intricate anatomy addressed by the diffusion catheter systems herein, emphasizing the necessity for positional flexibility and optimized diffusion characteristics in fluid-based therapy delivery. However, it will be appreciated that the potential configurations of actual vessels are practically infinite and that the layout shown in FIG. 1 is merely one example.

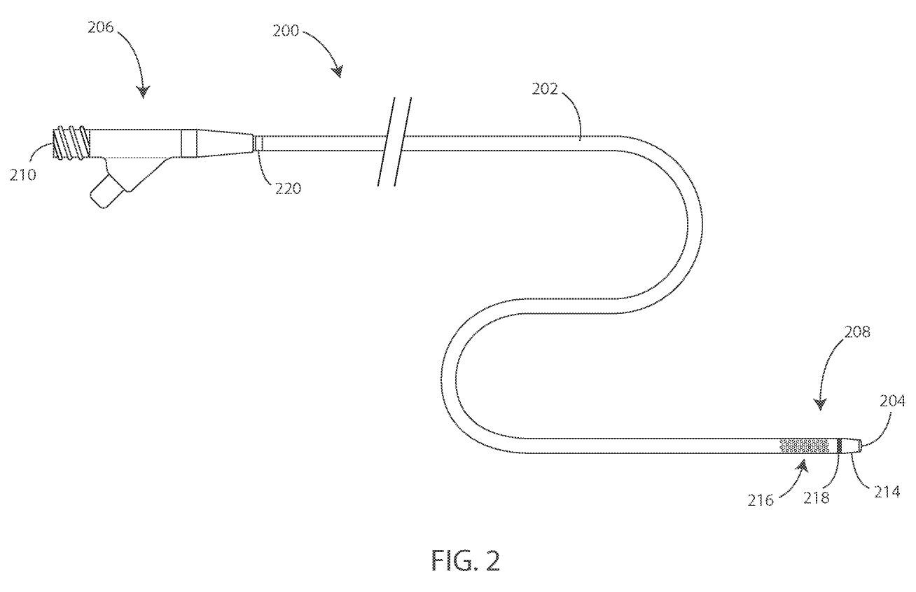

Referring now to FIG. 2, a schematic view of a microcatheter is shown in accordance with various embodiments herein. Specifically, FIG. 2 illustrates a microcatheter (200) herein designed for the targeted delivery of fluid-based therapies. The microcatheter includes a catheter shaft (202), which extends from a proximal end (206) to a distal end (208). The catheter shaft (202) can include a lumen therein. The catheter shaft (202) can be of various sizes. In some embodiments, the inner diameter of the catheter shaft (202) can be from about 0.013 inches (330 micrometers (μm)) or less to about 0.027 inches (685 μm) or more.

The catheter shaft (202) can be designed to offer both flexibility and structural integrity required for navigating the intricate pathways of the vasculature. The catheter shaft (202) can be constructed from various materials aimed at enhancing its mechanical properties, such as flexibility, torque control, and kink resistance. In some embodiments, the catheter shaft (202) can be fabricated using high-performance polymeric materials including polyether block amide (PEBAX), thermoplastic polyurethane (TPU), or polyimide. These polymers can be selected based on their mechanical properties, biocompatibility, and ability to be processed into thin-walled structures.

To further enhance the catheter shaft's (202) properties, it can include reinforced layers of braided or woven fibers. The fibers may be composed of materials such as stainless steel, nitinol, or high-strength polymer threads like Kevlar® or Vectran®. The braiding pattern of the fibers can be configured to provide optimized flexibility and strength, which aids in steering the catheter through tortuous vessel pathways and also to provide strength to contain potentially high pressures associated with injectate delivery. The fiber braiding can also improve torque transmission from the proximal end to the distal tip of the catheter, enhancing the maneuverability and control during medical procedures.

In another embodiment, the catheter shaft (202) may also include an inner liner or coating, made from materials such as polytetrafluoroethylene (PTFE) or other fluoropolymers, to facilitate smooth passage of therapeutic compositions through the catheter's lumen. This lining can minimize frictional resistance and prevent irregularities in the inner surface aiding in the delivery efficiency of fluid-based therapies.

In this example, the proximal end (206) is equipped with a threaded connector (210), which can provide a secure interface for attachment to a fluid therapy source. In some embodiments, a releasable locking mechanism can also be included near the proximal end (206) to allow the microcatheter (200) to be positionally locked in place relative to the seal element described further below. In some embodiments, one or more alignment marks (220) can be disposed on the microcatheter (200) at various points to aid in aligning the position of the microcatheter (200) with other system components. Moving toward the distal section, the catheter shaft (202) transitions into a tapered portion (214). In some embodiments, one or more radiopaque markers (218) can be included to aid in the visualization of positioning of the microcatheter (200) by a clinician.

The distal end (208) of the microcatheter (200) features an open end (204). Adjacent to this open end are a plurality of side apertures (216), strategically positioned along the catheter shaft. These apertures are geared towards improving the dispersion characteristics of the therapeutic fluid, thus facilitating its diffusion within the vessel and a more uniform distribution within the vessel. The tapered portion (214) located near the distal end (208) further complements this function, by potentially altering flow dynamics to support optimal diffusion efficacy. In specific, because the tapered portion (214) can serve to narrow down the inner diameter of the microcatheter (200) near the open end (204), it can facilitate at least partial occlusion of the open end (204) such that a therapeutic composition is preferentially passed out of the plurality of side apertures (216) instead of the open end (204). In some embodiments, the tapered portion (214) can reduce the inner diameter of the microcatheter (200) lumen by at least about 0.001, 0.003, 0.005, 0.007, 0.009 inches (25, 76, 127, 178, or 228 μm) over the length of the tapered portion (214), or an amount falling within a range between any of the foregoing.

The area or zone of the microcatheter (200) with the side aperture (216) can be about 0.25, 0.5, 1, 2, 3, 4, or 5 centimeters (0.098, 0.197, 0.394, 0.787, 1.181, 1.575, or 1.969 inches) in length or could have a length falling within a range between any of the foregoing. In some embodiments, the area or zone of the microcatheter (200) with the side aperture (216) can be the same material as other portions of the microcatheter (200). However, in some embodiments, the area or zone of the microcatheter (200) with the side aperture (216) can be formed of a different material. By way of example, in some embodiments, the area or zone of the microcatheter (200) with the side aperture (216) can be formed of a tubular portion of a metal, a composite, or a polymer.

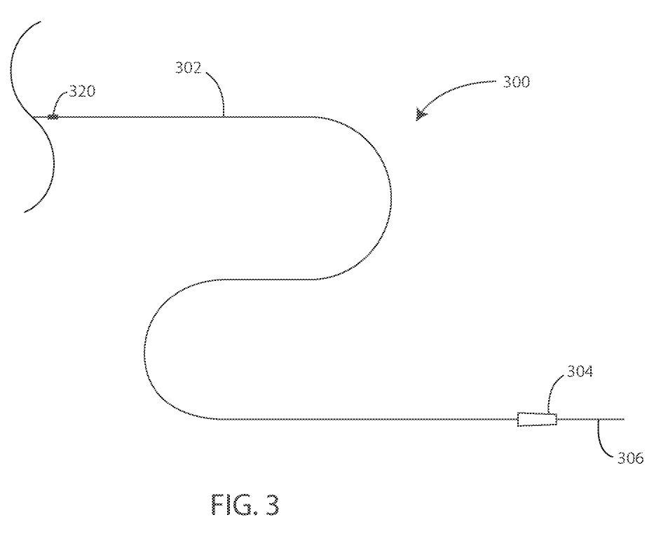

Referring now to FIG. 3, a schematic view is shown of a seal element in accordance with various embodiments herein. Specifically, FIG. 3 depicts a simplified schematic of a seal element (300) used with various embodiments of advanced diffusion catheter systems herein. The seal element (300) includes several key components, each serving specific functional purposes within the system. A shaft (302) extends longitudinally through the assembly, designed to pass through the microcatheter. The shaft provides structural support and acts as a guide for the other components of the seal element during its deployment and use.

Attached to the shaft is an occlusion member (304). The occlusion member (304) constitutes an integral part of the seal element, serving as the mechanism to at least partially occlude the open distal end of the microcatheter and thus regulate fluid flow directionality and enhance therapeutic agent distribution.

Various materials can be used to fabricate the occlusion member (304), encompassing biocompatible polymers such as silicone, polyurethane, and PTFE, as well as metallic compounds like stainless steel or nitinol, and various composites. The occlusion member can have many different shapes and forms, such as spherical, conical, frustoconical, cylindrical, or irregular shapes or the like. These shapes can be adapted to at least partially conform to the interior dimensions of various catheter lumens, such as in the area of the tapered portion of the microcatheter, ensuring at least partial occlusion of the open distal end of the microcatheter and causing fluid to flow out of the side apertures. In some embodiments, the occlusion member can take the form of a plug or stopper. In some embodiments, the occlusion member can have a textured surface to aid in engagement with the inner surface of the microcatheter.

The occlusion member's size may vary with diameters from approximately 0.5 millimeters (mm) (0.019 inches) or less to 3 mm (0.118 inches) or more, depending upon the specific design requirements of the catheter system, including the size of the open distal end of the microcatheter and the targeted vessel size.

It is important to note that in many cases complete occlusion of the open distal end is not required. Rather, occlusion should be sufficient to direct a desired amount of the therapeutic composition out of the side apertures. By way of example, in some embodiments, the occlusion member (304) can occlude the lumen inside the microcatheter near the open distal end by at least about 50, 60, 70, 80, 90, 95, 98, 99, or 100 percent, or an amount falling within a range between any of the foregoing.

Attachment of the occlusion member (304) onto the shaft (302) of the seal element can be achieved through methods such as adhesive bonding, mechanical attachment, or integrally molding it as a part of the shaft. These attachment methods can ensure a secure fit. However, for certain applications, a detachable or exchangeable occlusion member variant could be utilized, allowing for customization of the seal element to work with microcatheter of various sizes.

It will be appreciated that positioning of the occlusion member (304) can be important for its functionality. It can be oriented axially to precisely engage the open distal end of the microcatheter, whether by direct contact or through a coordinating distal sealing feature, such as a tapered portion, a stepped down portion, or the like. As described above, the occlusion effect redirects fluid flow from the open distal end to the side apertures along the catheter shaft, ensuring that the therapeutic agents are distributed evenly within the target vessel.

In some embodiments, one or more alignment marks (320) can be disposed on the seal element (300) at various points to aid in aligning the position of the seal element (300) with other system components.

In some embodiments, the occlusion member can be adjustable. In some embodiments, the occlusion member can include an expandable element, such as a balloon or other expandable occlusive device that can deploy within the lumen to achieve a complete or partial sealing effect. These alternatives provide flexibility in addressing diverse clinical scenarios while also catering to specific therapeutic needs or patient anatomies. Each configuration can facilitate adaptability and precision in therapeutic administration, thereby promoting improved treatment outcomes.

In some embodiments, the seal element can include a guide or guidewire portion (306). In FIG. 3, the guidewire portion (306) of the seal element is depicted distal to the occlusion member. The guidewire portion (306) of the seal element can facilitate repositioning of the microcatheter (200) within the vasculature of a patient. It allows the catheter to be maneuvered to various target therapy sites without necessitating the removal of the seal element. This adaptability is key, particularly when delivering multi-site therapies in a single procedure, as it eliminates the need for repetitive withdrawal and re-insertion of a guide wire, thereby minimizing procedure time, patient discomfort, and potential contamination issues. The guidewire portion (306) can be configured to allow properties similar to that of a traditional guidewire such as torque, pushability, and the like. In various embodiments, the guidewire portion (306) can be curved to facilitate steering it into desired target sites of the vasculature.

The materials used to fabricate the shaft 302 of the seal element, as well as the guidewire portion 306 (which could be the same or different from one another), can impact the performance of the system. The shaft 302 can be constructed from materials such as stainless steel, nitinol, or other metallic alloys exhibiting strength, flexibility, and corrosion resistance, key attributes required for navigating the tortuous paths of vascular systems. Stainless steel provides structural integrity and resistance to both mechanical forces and chemical interactions with possible therapeutic fluids. Nitinol, an alloy of nickel and titanium, offers superelastic properties allowing it to endure deformation while maintaining shape upon release, which is beneficial during intricate navigation. Alternatively, or in combination, the shaft (302) can be made from high-strength polymeric materials like polyetheretherketone (PEEK) or a polyimide. In some embodiments, a composite construction can be used, wherein a polymeric exterior encases a metallic core or braided reinforcement to enhance maneuverability while providing the necessary structural support to navigate complex vasculature. Similarly, the guidewire portion 306 can be constructed using flexible yet resilient materials such as the same or different materials than shaft (302). These material selections for both the shaft 302 and guidewire portion 306 can ensure that the seal element operates efficiently within the vascular environment, facilitating precise deployment and repositioning within the patient’s anatomy while minimizing trauma to vessel walls.

It will be appreciated that the ability to reposition the microcatheter without extracting the seal element and reinserting a guidewire holds significant advantages in terms of safety and efficiency, especially when the seal element has been exposed to a radioactive therapeutic composition. By reducing the frequency of inserting and withdrawing potentially contaminated components, the risk of radioactive exposure to both healthcare providers and patients is minimized. Additionally, the streamlined process supports a more efficient therapeutic workflow and potentially reduces the overall exposure of the patient to the therapeutic agents by allowing continuous and uninterrupted delivery across multiple sites. Thus, this innovative feature not only contributes to enhanced procedural precision but also addresses safety concerns relevant in the administration of radioactive therapies.

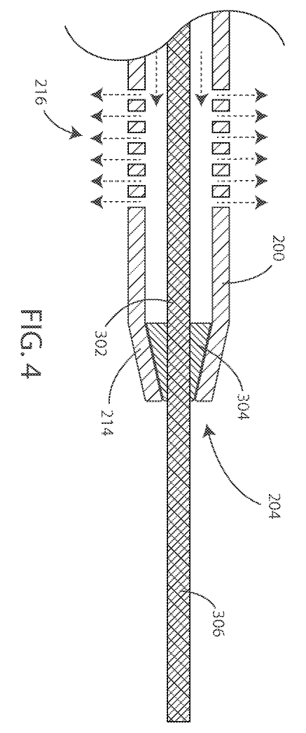

Referring now to FIG. 4, a cross-sectional view of a distal portion of a therapy delivery apparatus is shown in accordance with various embodiments herein. In specific, FIG. 4 provides a detailed cross-sectional view of the distal portion of a therapy delivery apparatus, specifically illustrating the configuration and interaction between the microcatheter 200, the occlusion member 304, and the guidewire portion 306.

As described previously, the microcatheter 200 features an open end 204. At or upstream from the open end 204 is a strategically designed tapered portion 214, which serves to narrow down the interior diameter of the microcatheter, thereby making it easier to occlude flow through the distal end of the microcatheter. It will be appreciated, however, that the tapered portion 214 is not necessarily required. By way of example, in some embodiments, a stepped down portion could be used to narrow down the interior diameter of the microcatheter. In some embodiments, an internal bump or similar structure could be used to narrow down the interior diameter of the microcatheter.

Embedded within the side walls of the microcatheter are side apertures 216. These apertures are configured to allow a therapeutic composition to flow out the sides of the microcatheter, thus enabling a more uniform distribution across the targeted treatment area. The arrangement of these apertures is optimized to improve the dispersion characteristics, mitigating risks associated with non-uniform delivery and promoting therapeutic efficacy.

The side apertures can take the form of slots, round holes, oval holes, polygonal holes, irregular holes, or other shapes. The geometry can be selected based on the type of fluid therapy, the size of particles within a suspension, and other factors.

In some embodiments, the side apertures can be configured to alter their shape and/or size in response to a threshold pressure within the microcatheter. This pressure responsive configuration allows the side apertures to regulate the flow rate there through. For example, the side apertures can be formed of a flexible material that assumes one diameter under a low pressure and assumes a larger diameter under a greater pressure. As another example, the side apertures can include flexible flaps (or a similar structure) that open under pressure. In some embodiments, the side apertures can be slot shaped to facilitate opening and closing. In some embodiments, the side apertures can include a compliant septum to facilitate opening and closing.

In operation, the insertion of a fluid into the microcatheter can be done in such a way that the pressure is increased inside the microcatheter to a point that exceeds the threshold pressure which can cause the side apertures to open or open wider, thus allowing fluid to flow out of the side apertures at a faster rate than would otherwise be possible if the side apertures did not open or open more widely.

In some embodiments, if a lower pressure is applied in the course of delivering the fluid therapy (injectate) the apertures would open a smaller amount causing the injectate delivery amounts to be lower and if a higher pressure is applied then the apertures would open a larger amount causing the injectate delivery amounts to be higher. As such, in some embodiments, the apertures can effectively act similarly to valves that open and close in response to the applied pressure.

In various embodiments, the side apertures are arranged around the circumference of the microcatheter shaft to promote multidirectional diffusion. This circumferential configuration allows the therapeutic agent to be emitted evenly towards the surrounding vessel walls. In some embodiments, the side apertures are arranged evenly and/or symmetrically around the circumference of the microcatheter shaft. In other embodiments, the side apertures are arranged non-symmetrically or biased to a particular side.

FIG. 4 further details the integral components of the seal element, including a longitudinally extending shaft 302 which traverses through the microcatheter's lumen. The shaft provides structural integrity and guidance for the seal element components.

Attached to the shaft is the occlusion member 304, which is designed to engage the distal open end of the microcatheter. This key component can partially or fully occlude the open end to control fluid flow, effectively redirecting it through the side apertures. This redirection enhances diffusion and dispersion of the therapeutic composition within the target vessel to which it is delivered. Distal to the occlusion member is the guidewire portion 306, which functions to aid in accurate positioning and stability of the seal element within the catheter.

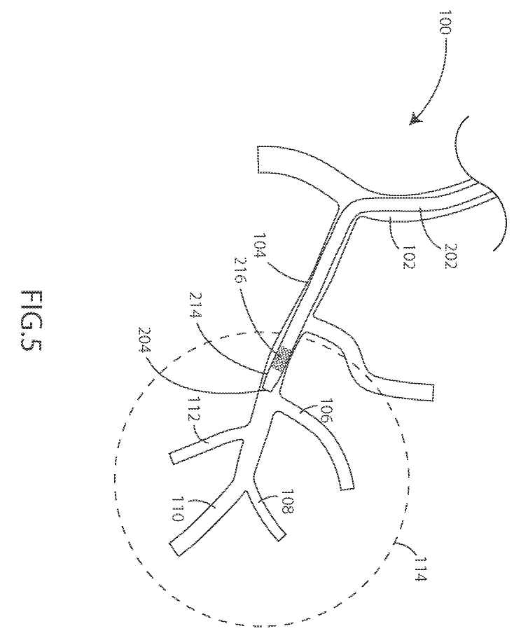

FIGS. 5-9 illustrate the deployment of components herein within vessels, the delivery of a therapeutic composition within vessels, and the repositioning of components herein within vessels. Referring now to FIG. 5, a schematic view is shown of a microcatheter within vessel anatomy in accordance with various embodiments herein. In FIG. 5, the schematic view illustrates the shaft (202) of a microcatheter positioned within a complex vessel network for targeted delivery of fluid-based therapies. The depiction shows the microcatheter traversing through the small diameter access vessel (102), which serves as an entry path into the vascular anatomy. A side branch vessel (104) extends from the main vessel, leading to several interconnected feeder vessels: the first feeder vessel (106), the second feeder vessel (108), the third feeder vessel (110), and the fourth feeder vessel (112).

The shaft (202) of the microcatheter is positioned within this network of vessels, orienting its distal end toward the target area (114), delineated by a dashed circle, indicating the targeted region for therapy application. In this example, the catheter shaft (202) comprises a tapered portion (214), an open end (204), and side apertures (216) along the shaft. These apertures enable multidirectional diffusion, and specifically allow a therapeutic composition to pass out to the sides of the microcatheter, promoting even distribution of the therapeutic agent across the target site. This configuration is particularly advantageous in achieving uniform treatment delivery within intricate vessel networks.

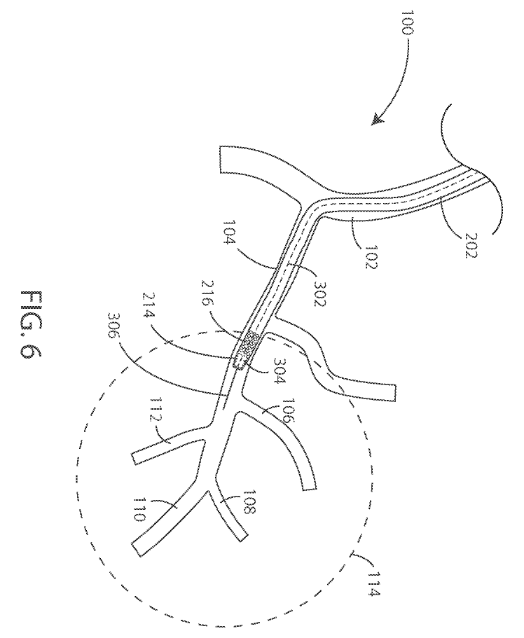

Referring now to FIG. 6, a schematic view is shown of an apparatus herein within vessel anatomy in accordance with various embodiments herein. In this example, a sealing element is positioned within the microcatheter.

As before, the apparatus passes through a small diameter access vessel (102) which serves as a conduit into a branching network. The catheter shaft (202) traverses through the access vessel and reaches into a side branch vessel (104). The branch further divides into first (106), second (108), third (110), and fourth (112) feeder vessels, each extending into distinct paths within the network.

The catheter shaft (202) features a tapered portion (214) and multiple side apertures (216), arranged to ensure optimal fluid distribution. FIG. 6 also shows the shaft (302) of the sealing element extending through the catheter shaft, and supporting an occlusion member (304) located near the distal end. The occlusion member (304) interacts with the open end of the microcatheter or with the interior of the microcatheter, such as at the tapered portion 214, to facilitate occlusion of the lumen of the microcatheter at or upstream from the open end to cause redirection of fluid flow through the side apertures. Completing the system is the guidewire portion (306) of the sealing element facilitating maneuverability of the system.

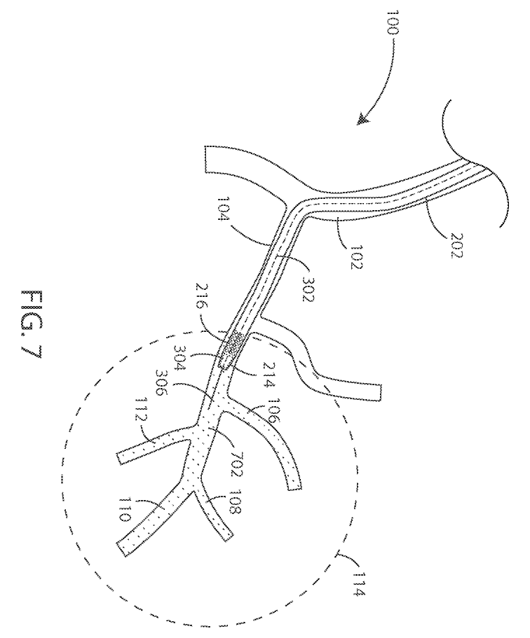

Referring now to FIG. 7, a schematic view is shown of an apparatus herein within vessel anatomy in accordance with various embodiments herein. In specific, FIG. 7 illustrates the shaft (202) of the microcatheter and the sealing element being positioned within the vasculature and a therapeutic composition (702) to be delivered to the targeted area. As before, the catheter shaft (202) traverses a small diameter access vessel (102) which branches into a side branch vessel (104), further diverging into a network of feeder vessels: the first feeder vessel (106), the second feeder vessel (108), the third feeder vessel (110), and the fourth feeder vessel (112). Further, as before, the catheter shaft (202) transitions into a tapered portion (214) as it approaches the designated target area (114), which is defined by a dashed circle. Positioned along the catheter shaft (202) are multiple side apertures (216), which aid in facilitating multidirectional diffusion of the therapeutic composition (702). The shaft (302) of the sealing element supports an occlusion member (304) near the distal end. The occlusion member interacts with the open end, managing fluid flow effectively. The guidewire portion (306) aids in ensuring accurate positioning within the complex vessel anatomy and repositioning.

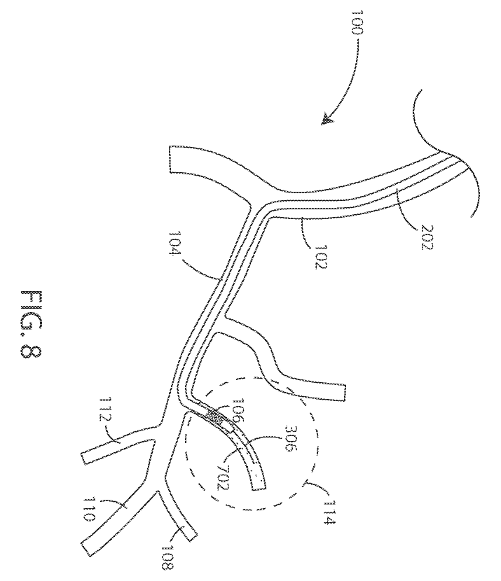

Referring now to FIG. 8, a schematic view is shown of an apparatus herein within vessel anatomy in accordance with various embodiments herein. In specific, FIG. 8 illustrates a scenario where an apparatus configured for fluid-based therapy delivery herein may be repositioned within a complex vascular architecture. As before, a small diameter access vessel (102) is depicted which branches into a side branch vessel (104), which further branches into multiple feeder vessels, specifically the first feeder vessel (106), second feeder vessel (108), third feeder vessel (110), and fourth feeder vessel (112).

As before, the catheter shaft (202) of the microcatheter is shown, but in this view it is illustrated passing into the first feeder vessel (106). As such, the distal segment of the catheter shaft (202) approaches a new designated target area (114) within the vascular network, marked by a dashed circle, representing the therapeutic focus area for fluid dispersal at a new site. The guidewire portion (306) of the sealing element is depicted passing into the first feeder vessel (106) and assists in repositioning of the device within the vessel network. The therapeutic composition (702) is shown being delivered within the first feeder vessel 106. FIG. 8 highlights the role that the sealing element and specifically the guidewire portion (306) thereof plays in repositioning of the microcatheter within the vasculature so that a new target area (114) can be treated.

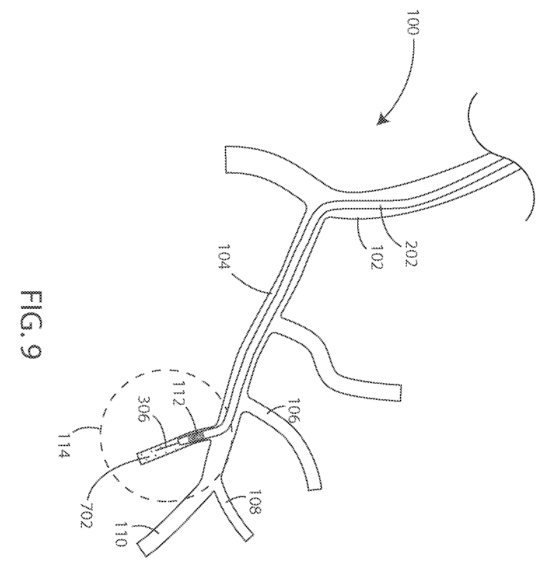

It will be appreciated that microcatheters herein may be repositioned multiple times such that multiple sites within the vasculature can be treated. Referring now to FIG. 9, a schematic view is shown of an apparatus herein within vessel anatomy in accordance with various embodiments herein. As before, a small diameter access vessel (102) provides a pathway for the catheter system into the broader vessel architecture including side branch vessel (104) which branches into the first feeder vessel (106), the second feeder vessel (108), the third feeder vessel (110), and the fourth feeder vessel (112).

FIG. 9 shows the catheter shaft (202) traversing the small diameter access vessel (102), the side branch vessel (104), and passing into the fourth feeder vessel (112) to allow treatment of a new designated target area (114), highlighted with a dashed circle, signifying the region intended for precise therapeutic delivery.

The guidewire portion (306) of the sealing element is depicted passing into the fourth feeder vessel (112) and assists in repositioning of the device within the vessel network. The therapeutic composition (702) is shown being delivered within the fourth feeder vessel (112). FIG. 9 highlights the role that the sealing element and specifically the guidewire portion (306) thereof plays in repositioning of the microcatheter within the vasculature so that a new target area (114) can be treated without requiring reinsertion of a guidewire.

The side wall of the microcatheter can be formed using various constructions and various materials. The side wall of the microcatheter can include multiple layers, with different layers providing different functional properties. In some embodiments, a braided structure can be included within the wall of the microcatheter. In areas where side apertures are included, it can be advantageous to position the side apertures so as to maintain as much strength of the side wall as possible.

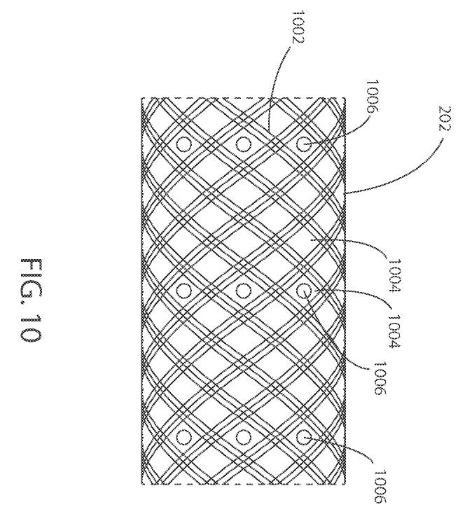

Referring now to FIG. 10, a schematic view of a microcatheter wall is shown in accordance with various embodiments herein. The catheter shaft (202) is shown with an integrated braided structure (1002) therein, which contributes to the overall strength, kink resistance, and pushability of the catheter, which aids in navigating complex vascular networks.

Interspersed within the braided pattern are open gaps (1004) between adjacent braid strands or braid bundles. Apertures (1006) can be formed so that they are within the open gaps (1004), which allows the braided structure to maintain its structural integrity. The apertures (1006) can be formed using various techniques including, but not limited to, drilling, piercing, machining, EDM (electrical discharge machining), laser cutting, molding, and the like. The diameter of apertures (1006) can vary. In some embodiments, the diameter of apertures (1006) herein can be greater than the diameter of microparticles used with therapeutic suspensions herein. In some embodiments, the apertures (1006) can be about 30, 40, 50, 75, 100, 125, 150, 175, 200, 250 μm (0.0012, 0.0016, 0.0020, 0.0030, 0.0039, 0.0049, 0.0059, 0.0069, 0.0079, 0.0098 inches) or larger, or a size falling within a range between any of the foregoing. In some embodiments, the apertures (1006) can be about 127 μm or about 0.005 inches in diameter. The side apertures (1006) can be the same or similar shapes and sizes as those of the side apertures (216) of embodiments discussed above with respect to FIGS. 2 and 4-9 above.

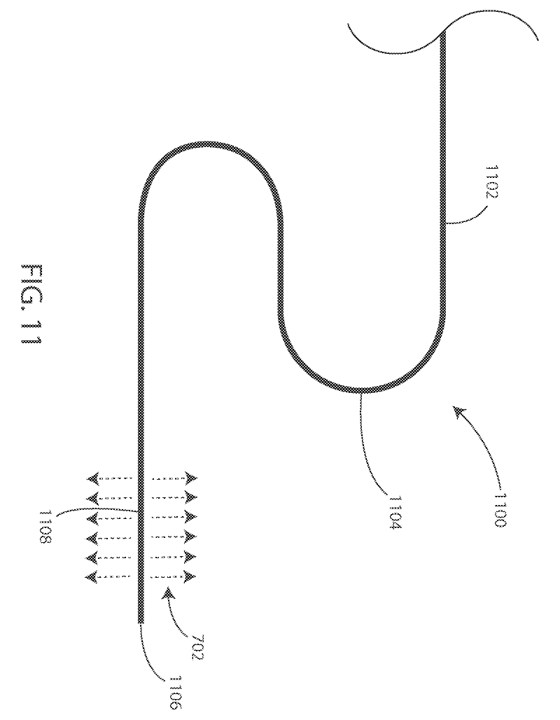

In some embodiments, therapeutic compositions can be delivered through devices other than directly through a microcatheter such as that illustrated previously herein. Referring now to FIG. 11, a schematic view of an insert wire (1100) is shown in accordance with various embodiments herein. Specifically, FIG. 11 illustrates the configuration and functionality of an insert wire (1100) designed for the delivery of therapeutic compositions (702) into a target vessel. The insert wire (1100) includes a flexible hollow shaft (1102), which is constructed to provide both a passageway for the delivery of a therapeutic composition as well as the necessary maneuverability within the vessel. In some embodiments, the insert wire (1100) can be used entirely separately from a microcatheter, but in other embodiments the insert wire (1100) can be used in conjunction with a microcatheter, such as with the insert wire passing through the microcatheter that provides proximal support to assist navigation of the insert wire (1100) to a proper treatment location.

In some embodiments, the flexible hollow shaft (1102) can be formed with a hollow hypotube (or hypodermic tube), which could be metal, polymer, a composition, or a combination of different materials. In some embodiments, the flexible hollow shaft (1102) is enhanced with a reinforcement layer (1104) that ensures structural integrity, preventing kinking and maintaining fluid flow efficiency. The reinforcement layer (1104) can be made from materials that balance both strength and flexibility, potentially involving braided or non-braided structures, and potentially including polymers, metals, composites, or the like.

In some embodiments, the flexible hollow shaft (1102) of the insert wire (1100) can be constructed from a metal hypotube and the shaft can include surface features that significantly bolster its flexibility. These features can ensure that the shaft can navigate the intricate and convoluted pathways often encountered in vascular systems without compromising structural integrity or the inner lumen through which therapeutic compositions are delivered. By way of example, the hypotube can include slots (elongated openings) placed along the length of the hollow shaft 1102. The presence of these slots contributes to the adaptability and flexibility of the insert wire 1100 by enabling localized deformation of the shaft when navigating sharp bends or tortuous pathways within the vasculature. Pushability of the insert wire (1100) can be enhanced by the ability of the slots to accommodate compression forces along the wire's axis, allowing clinicians to exert directional force without undue bending or kinking.

The insert wire (1100) can include a fully or at least partially closed distal end (1106). The fully or partially closed distal end (1106) can be configured to minimize dead space and direct the therapeutic composition (702) toward a diffusion tip, which is a portion of the insert wire (1100) that comprises a series of side apertures (1108). These apertures allow the therapeutic agents to diffuse outward from the insert wire (1100) to the side, facilitating uniform distribution within the target vessel.

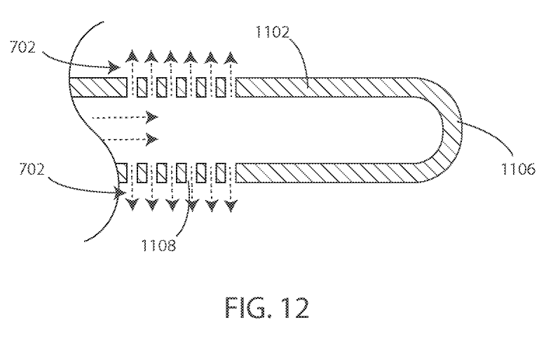

Referring now to FIG. 12, a schematic cross-sectional view is shown of a distal end of an insert wire in accordance with various embodiments herein. FIG. 12 shows the flexible hollow shaft (1102) and the therapeutic composition (702) passing out of the same. In specific, in this configuration the therapeutic composition (702) is transported through the lumen of the flexible hollow shaft and directed toward the distal end. However, since the end is characterized by a fully or partially closed distal end (1106), designed to minimize dead space, the flow of the therapeutic composition is directed out of the insert wire through the multiple side apertures (1108).

The side apertures (1108) can be the same or similar shapes and sizes as those of the side apertures (216) of embodiments discussed above with respect to FIGS. 2 and 4-9 above.

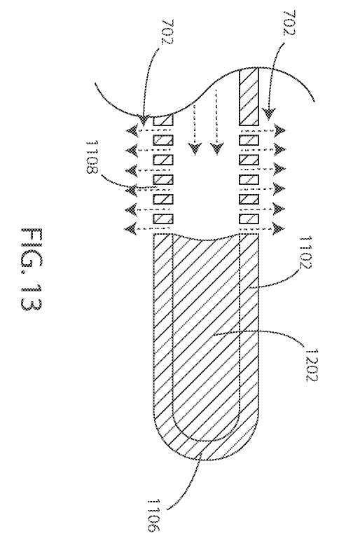

As referenced above, in some embodiments, the insert wire and, specifically, the distal end thereof can be configured to minimize dead space therein. Referring now to FIG. 13, a schematic cross-sectional view is shown of a distal end of an insert wire in accordance with various embodiments herein. As before, the insert wire is designed for delivery of therapeutic compositions (702) into target regions. As before, a flexible hollow shaft (1102) is shown which provides for the passage of the therapeutic composition (702) toward the target site. The shaft (1102) is constructed to maintain flow integrity while offering the necessary flexibility for navigating intricate vascular pathways. The distal end of the shaft (1102) is characterized by a closed (fully or partially) distal end (1106) adjacent to a diffusion tip which features multiple side apertures (1108) configured to facilitate multidirectional diffusion of the therapeutic composition.

In the example of FIG. 13, the interior of the distal end of the insert wire incorporates a filled portion (1202), which aids in both minimizing dead space (ensuring that minimal volumes of the therapeutic composition are trapped within unused sections of the device) while guiding the flow of the therapeutic composition toward the side apertures (1108). In some embodiments, the filled portion (1202) is contoured to directly conform to the internal geometry of the diffusion tip, ensuring there is a streamlined path for fluid passage and minimizing any potential disruption to the flow dynamics as the therapeutic composition (702) is directed toward and through the side apertures (1108). This assists in achieving the necessary conditions for optimal diffusion and distribution of the therapeutic agent within the designated target vessel, thereby enhancing overall treatment efficacy.

Potential materials for the filled portion (1202) include various polymers such as polyethylene and polyurethane, which can provide a lightweight and malleable solution, allowing shaping to best suit specific application needs. In alternative configurations, the filled portion (1202) can be fabricated from metallic materials such as titanium, stainless steel, or the like. In some embodiments, the filled portion (1202) can be formed from an adhesive material. Additionally, in some embodiments the material used for the filled portion (1202) may also incorporate radiopaque markers to facilitate visualization under fluoroscopy, allowing clinicians to accurately position and monitor the function of the insert wire during procedures.

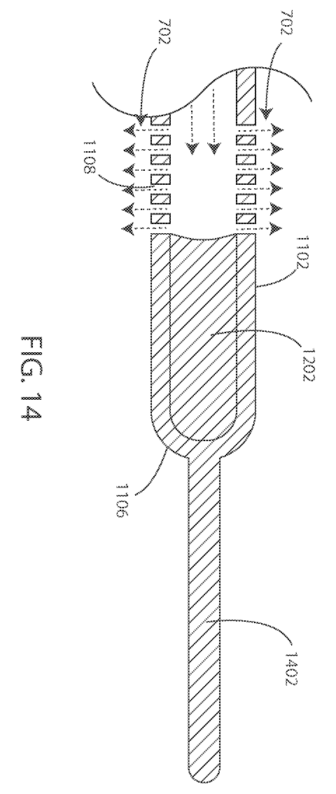

As described above, it can be highly desirable to reposition components of the system without removing components from the vasculature of the patient. Referring now to FIG. 14, a schematic cross-sectional view is shown of a distal end of an insert wire in accordance with various embodiments herein. The insert wire includes a flexible hollow shaft (1102), providing a channel for the targeted deployment of therapy, along with a filled end (1202) to minimize dead space, and a closed distal end (1106). The distal end of the insert wire includes multiple strategically arranged side apertures (1108).

Repositioning capabilities of the insert wire are enhanced by the inclusion of a steering element (1402), extending beyond the closed distal end (1106). This steering element can aid in navigating the vessel network, allowing for repositioning of the insert wire. The steering element (1402) can include similar materials and shapes as those described with respect to the guidewire portion 306 of the seal element 300 described above.

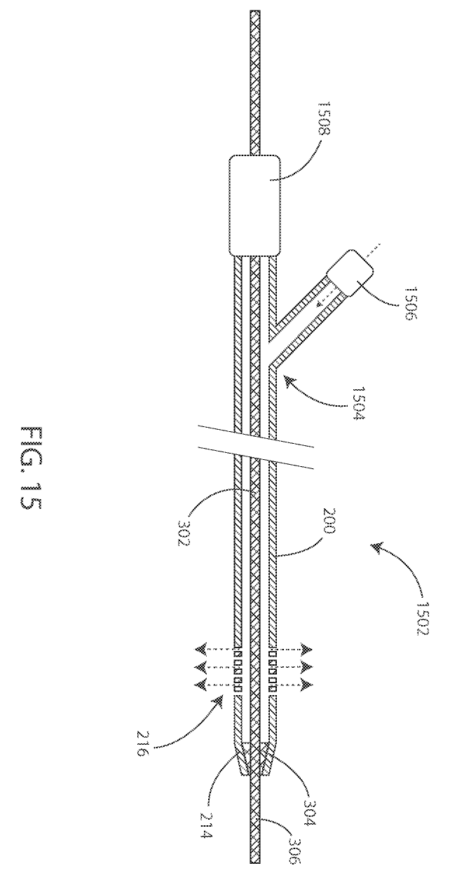

Various options are contemplated herein for how a fluid can enter and/or pass through the apparatus herein. Referring now to FIG. 15, a schematic partial cross-sectional view of a therapy delivery apparatus (1502) is shown consistent with various embodiments herein. Consistent with other embodiments described herein, a longitudinally extending shaft (302) traverses through the catheter shaft (200) and into a designed tapered portion (214) at the distal end of the catheter. An occlusion member (304), positioned near the distal end, interfaces with a designed tapered portion (214) of the catheter to at least partially obstruct the open end thereby directing therapeutic compositions toward strategically located side apertures (216). The apertures (216) facilitate multidirectional diffusion and uniform distribution of the therapeutic agent within the target vessel. A guidewire portion (306) extends beyond the occlusion member, enhancing the apparatus's navigability and repositioning capability.

In this example, the apparatus is designed for fluid delivery through a microcatheter system that includes a Y manifold (1504). The Y manifold (1504) is connected to a proximal end of the catheter shaft (200). Adjacent to the Y manifold (1504) is a side port (1506), though which the fluid can be injected or otherwise infused. A Touhy-Borst port (1508) can be positioned at the proximal end providing a mechanism for securing the shaft (302) or another portion of the seal element, ensuring they remain in place during the procedure.

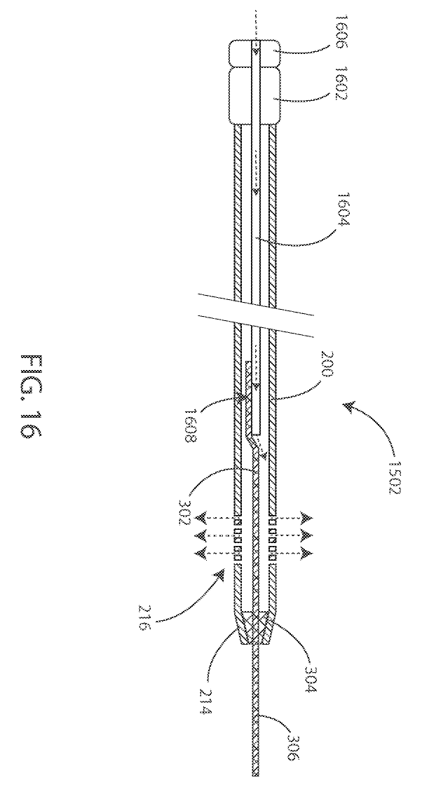

However, it will be appreciated that other configurations are also contemplated herein. As an example of another embodiments, FIG. 16 shows a schematic partial cross-sectional view of a therapy delivery apparatus (1502) consistent with various other embodiments herein. As before, the apparatus (1502) is designed for the delivery of fluid-based therapies through a microcatheter system. FIG. 16 shows many of the same components as does FIG. 15 including guidewire portion (306), occlusion member (304), tapered portion (214), side apertures (216), and shaft (302).

However, in the configuration of FIG. 16, a female Luer fitting (1602) is positioned proximally, providing a connection interface for a male Luer fitting (1606) through which a fluid can be provided. A hypotube (1604) is connected to and extends distally from the female Luer fitting (1602) forming a pathway through which therapeutic fluids are channeled. In operation, then, the fluid can be injected or otherwise infused passing through male Luer fitting (1606) and through the hypotube (1604) and at least part-way through the catheter shaft (200) before exiting the hypotube (1604) through an end portion thereof (such as through an aperture at the end of the hypotube) and then traveling some distance through the lumen of the catheter shaft (200) before passing out of the side apertures (216). In some embodiments, the hypotube (1604) can be connected to the shaft (302) of a seal element at an attachment point (1608). In various embodiments, the hypotube (1604) can extend to or nearly to the position of the side apertures (216), but in other embodiments, the hypotube (1604) can stop before the position of the side apertures (216) such as millimeters or centimeters before the position of the side apertures (216).

While not intending to be bound by theory, the embodiment of FIG. 16 can offer advantages in that there is less dead space, better control of the seal element, and more robust connections to prevent leaks. In addition, the Luer fitting (or other fitting) can be attached to the hypotube at a length that aligns the seal element with the tapered portion (214) at the distal end of the catheter shaft (200). In some embodiments, other types of fittings can be used instead of Luer fittings.

### Methods

Many different methods are contemplated herein, including, but not limited to, methods of making, methods of using, methods of treating a patient, methods of delivering a therapeutic agent to a vessel of a patient, and the like. Aspects of system/device operation described elsewhere herein can be performed as operations of one or more methods in accordance with various embodiments herein.

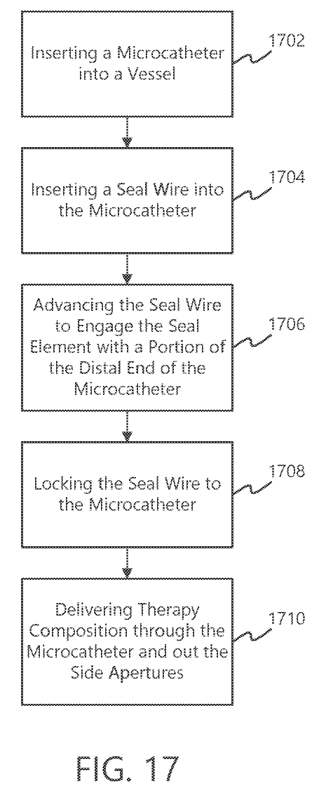

Referring now to FIG. 17, a flow chart is shown of operations of a method in accordance with various embodiments herein. In an embodiment, a method of delivering a fluid-based therapy into a vessel of a patient can include an operation of inserting a microcatheter into the vessel of the patient 1702, wherein the microcatheter comprises a plurality of side apertures near its distal end. The method can further include an operation of inserting a seal wire into the microcatheter 1704, wherein the seal wire comprises a seal element. The method can further include advancing the seal wire to engage the seal element 1706 with a portion of a distal end of the microcatheter. The method can further include locking the seal wire to the microcatheter 1708, and delivering therapy fluid through the microcatheter and out the side apertures 1710 into the vessel. It will be appreciated, however, that in some embodiments operation 1708 can be omitted (e.g., the seal wire is not locked in position relative to the microcatheter). In some embodiments, the method can further include repositioning the microcatheter and seal wire within the vessel for delivery of therapy to multiple sites without removing the seal wire from the microcatheter. In some embodiments of the method, the fluid-based therapy comprises a cancer therapy. In an embodiment of the method, the fluid-based therapy comprises radioactive microspheres in a carrier fluid.

### Microspheres

Many different types of fluid-based therapies can be delivered with devices and systems herein. However, in some embodiments, the fluid of the therapy can specifically be a suspension of microspheres in a carrier fluid, such as used to treat cancer. Microspheres herein can include those with a combination of yttria, alumina, and silica. By way of example, in some embodiments, microspheres herein can include Y₂O₃-Al₂O₃-SiO₂ in a 40:20:40 wt. % ratio. It will be appreciated however, that other types of microspheres are also contemplated herein including those with other materials.

In some embodiments, microspheres can be prepared by combining yttrium-89 with alumina and silica, in some cases also using a flame spheroidization method, and using neutron bombardment to convert Y-89 into the beta emitting radioisotope Y-90. In various embodiments, the amount of beta radiation can exceed 400, 600, 800, 1000, 1500, 2000, 2500, 3000, 4000, 5000, 6000, 7000, 8000, 9000, 10000, or even 12000 Becquerel (Bq) per sphere or higher at the time of activity calibration (recognizing that the amount of radiation will drop after that point as the Y-90 radioisotope decays). In some embodiments, the microspheres can be provided in a vial with activity of 3 Gigabecquerel (GBq) or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, 0.1, 0.05, or 0.01 GBq, or less at calibration time, or an amount falling within a range between any of the foregoing. It will be appreciated that the use of other isotopes is also contemplated herein.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gray (Gy), 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 15 micrometers (μm) to about 35 μm. However, in some embodiments the microspheres can be somewhat smaller or larger.

The density of the microspheres can be quite high. In some embodiments, the density of the microspheres can be above 1.4, 1.8, 2, 2.5, or 3 grams/milliliter (g/mL), such as from 3.1 to 3.5 g/mL, or about 3.3 g/mL. By comparison, the density of water at room temperature is about 0.9978 g/mL. As such, the density of microspheres is much higher than an exemplary carrier fluid such as a saline solution which influences how readily such microspheres can settle out of a suspension.

It should be noted that, as used in this specification and the appended claims, the singular forms "a," "an," and "the" include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase "configured" can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An apparatus for delivering fluid-based therapies comprising:
a microcatheter, the microcatheter comprising
a catheter shaft;
a lumen, wherein the lumen is within the catheter shaft;
an open end, wherein the open end is at a distal end of the catheter shaft; and
a plurality of side apertures;
wherein the plurality of side apertures are disposed within a side wall of the catheter shaft;
wherein the plurality of side apertures allow for a fluid to pass from the lumen to the outside of the catheter shaft;
a seal element, wherein the seal element is configured to pass through the lumen of the microcatheter, the seal element comprising
a shaft; and
an occlusion member, wherein the occlusion member is attached to the shaft and is configured to at least partially occlude the open end of the microcatheter directing fluid flow through the plurality of side apertures.

2. The apparatus for delivering fluid-based therapies of claim 1, the microcatheter further comprising a distal sealing feature.

3. The apparatus for delivering fluid-based therapies of claim 2, the distal sealing feature comprising a tapered portion, wherein the tapered portion is disposed adjacent the open end and narrows the lumen.

4. The apparatus for delivering fluid-based therapies of claim 3, wherein the occlusion member is configured to engage with the tapered portion.

5. The apparatus for delivering fluid-based therapies of claim 2, the distal sealing feature comprising a stepped down portion.

6. The apparatus for delivering fluid-based therapies of claim 2, the distal sealing feature comprising an internal bump.

7. The apparatus for delivering fluid-based therapies of claim 1, the occlusion member comprising a stopper.

8. The apparatus for delivering fluid-based therapies of claim 1, the occlusion member comprising a textured surface, wherein the textured surface is configured to enhance engagement with an interior surface of the open end of the microcatheter.

9. The apparatus for delivering fluid-based therapies of claim 1, wherein the plurality of side apertures allow for diffusion and/or homogenization of the therapy within a target vessel after passing out of the microcatheter.

10. The apparatus for delivering fluid-based therapies of claim 1, wherein the plurality of side apertures are configured to change shape and/or size at a threshold pressure.

11. The apparatus for delivering fluid-based therapies of claim 1,
wherein a portion of the shaft extends beyond the occlusion member; and
wherein the portion of the shaft extending beyond the stopper comprises a steering element configured to aid in repositioning of the microcatheter.

12. The apparatus for delivering fluid-based therapies of claim 11, wherein the microcatheter is configured to be repositionable within a vessel of a subject to allow for movement to multiple therapy delivery sites without removal of the seal element from the microcatheter.

13. The apparatus for delivering fluid-based therapies of claim 1, the catheter shaft comprising:
a shaft wall, the shaft wall comprising a non-braided structure, wherein the non-braided structure is disposed within the shaft wall.

14. The apparatus for delivering fluid-based therapies of claim 1, the catheter shaft comprising:
a shaft wall, the shaft wall comprising a braided structure, wherein the braided structure is disposed within the shaft wall.

15. The apparatus for delivering fluid-based therapies of claim 14, wherein the plurality of side apertures are disposed within open gaps of the braided structure.

16. The apparatus for delivering fluid-based therapies of claim 1, further comprising:
a proximal fitting; and
a hypotube;
wherein the hypotube is connected to the proximal fitting and the seal element; and
wherein the hypotube is configured to receive the fluid and guide the same at least partway through the microcatheter.

17. An insert wire for delivering fluid-based therapies to a vessel of a patient comprising:
a flexible hollow shaft; and
a diffusion tip, wherein the diffusion tip is at a distal end of the flexible hollow shaft, the diffusion tip comprising
a plurality of side apertures; and
an at least partially closed distal end;
a connection hub;
wherein the connection hub is disposed at a proximal end of the insert wire; and
wherein the connection hub is configured to allow connection of the insert wire to a fluid source.

18. The insert wire of claim 17, further comprising a steering element, wherein the steering element extends beyond the at least partially closed distal end.

19. The insert wire of claim 17, wherein the insert wire allows repositioning of a microcatheter without removal of the insert wire.

20. The insert wire of claim 17, wherein the at least partially closed distal end is configured to minimize dead space at the end of the flexible hollow shaft.
